(19) United States
(12) Patent Application Publication
(10) Pub. No.: US 2014/0261419 A1
(43) Pub. Date: Sep. 18, 2014

(54) WATER CHAMBER FOR A RESPIRATORY THERAPY APPARATUS AND A PARTITION OF THE WATER CHAMBER

(71) Applicant: APEX MEDICAL CORP., New Taipei City (TW)

(72) Inventor: Chia-Hsiang Hsiao, New Taipei City (TW)

(73) Assignee: APEX MEDICAL CORP., New Taipei City (TW)

(21) Appl. No.: 13/836,152

(22) Filed: Mar. 15, 2013

(51) Int. Cl. A61M 16/16

(52) U.S. Cl. CPC A61M 16/16
USPC 128/204.14

(57) ABSTRACT

A water chamber for a respiratory therapy apparatus has a base casing, a cover casing and a partition. The partition makes the air flows downward, turns around and then goes up to prolong the route and the time of the air passing through the water chamber. Therefore, the air can be saturated with moisture sufficiently to increase the air humidity. When the air is passing through the water chamber, the air is divided into two parts to each respectively enter a first tube and a second tube. The audio frequencies caused by the two parts of the air are changed due to a change of a cross sectional area. When the two parts of the air converge, the audio frequency is changed again. The partition makes the air in different portions of the water chamber have different audio frequencies, thereby reducing the resonance to eliminate noise.

### BACKGROUND OF THE INVENTION

1. Field of the Invention

The present invention relates to a water chamber for a respiratory therapy apparatus and a partition of the water chamber, especially to a water chamber for a respiratory therapy apparatus and its partition that can increase air humidity and eliminate noise.

2. Description of the Prior Arts

Obstructive Sleep Apnea (OSA) is a common syndrome. When the patient with obstructive sleep apnea falls asleep, the upper respiratory tract may fall to obstruct the breathing, which severely affects the sleep quality. The patient can do exercise, take medication, or have surgery to relieve the symptom of the obstructive sleep apnea, or use a respiratory therapy apparatus to assist in sleeping.

The respiratory therapy apparatus comprises an air output device, a connecting tube, and a mask. The connecting tube connects between the air output device and the mask. The mask covers the face of the patient. The air output device outputs air to the mask via the connecting tube, and the air output device opens the upper respiratory tract of the patient by continuous positive airway pressure (CPAP) to assist the patient in breathing.

The respiratory therapy apparatus further has a water chamber mounted between the air output device and the connecting tube. The water chamber contains water inside. The air from the air output device enters the connecting tube after passing through the water chamber, thereby increasing the humidity of the air to prevent the air from being too dry and causing discomfort of the respiratory tract of the patient.

However, the conventional water chamber for the respiratory therapy apparatus has the following shortcomings.

First, the conventional water chamber is just a simple hollow box. The air passing through the conventional water chamber does not encounter any obstruction such that the air soon flows out to the connecting tube. Because the air stays in the conventional water chamber just for a very short time, the air is not sufficiently saturated with the moisture. Thus, the air filling into the mask may not contain enough humidity.

Second, similarly, because the conventional water chamber is a simple hollow box, the air passes through the conventional water chamber in a same speed via a single passage. Therefore, audio frequencies caused by the air are similar, thereby easily generating resonance, and the resonance may cause noise. The respiratory therapy apparatus is used to assist in sleeping, but paradoxically the noise disturbs sleep.

Third, when the conventional water chamber is in use, breathing of the user may generate a pressure, which makes the water in the water chamber flow back to the air output device, and the air output device is damaged therefore.

To overcome the shortcomings, the present invention provides a water chamber for a respiratory therapy apparatus and a partition of the water chamber to mitigate or obviate the aforementioned problems.

### SUMMARY OF THE INVENTION

The main objective of the present invention is to provide a water chamber for a respiratory therapy apparatus and a partition of the water chamber that can increase air humidity and eliminate noise.

The partition of the water chamber for a respiratory therapy apparatus has a panel body, a first tube, a second tube and a panel outlet. The panel body has an inlet end and an outlet end. The first tube and the second tube are formed on a bottom surface of the panel body. The first tube is shorter than the second tube. The first tube is nearer to the inlet end than the second tube. The second tube is nearer to the outlet end than the first tube. The panel outlet is formed through the panel body and is adjacent to the outlet end.

The water chamber for a respiratory therapy apparatus has a base casing, a cover casing, an inner space and the partition as described. The cover casing is mounted on the base casing, and has a casing inlet, a casing outlet and a casing panel. The inner space is formed between the base casing and the cover casing. The partition is mounted between the base casing and the cover casing, and divides the inner space into an upper space and a lower space. The casing panel of the cover casing further divides the upper space into an inlet space and an outlet space. The casing inlet communicates with the inlet space and corresponds to the inlet end of the panel body. The casing outlet communicates with the outlet space and corresponds to the outlet end of the panel body. The first tube and the second tube communicate with the inlet space and the lower space. The panel outlet communicates with the outlet space and the lower space, and corresponds to the casing outlet.

Because the water chamber has the partition mounted inside, the partition can prolong the route of the air passing through the water chamber, and the partition can also change the speed and the direction of the air passing through the water chamber as described below. The air enters the upper space of the water chamber from the casing inlet. Then the air is divided into two parts each respectively entering the first tube and the second tube to reach the lower space. Because the second tube is longer than the first tube, the air from the second tube passes through a relative longer route, which makes the speed of the air from the second tube relatively slower. According to Bernoulli's Principle, a fluid with slower speed has higher pressure. Besides, air with higher pressure must flow toward air with lower pressure. Thus, the part of the air from the second tube with relative higher pressure flows toward the part of the air from the first tube with relative lower pressure. Then, the two parts of the air converge and change the flowing direction. Afterwards, the air flows along an inside surface of the base casing to flow around, and then the air flows upward to the panel outlet and the casing outlet in sequence. Finally, the air flows into a connecting tube and a mask. The partition makes the air go downward, turn around and then go upward to prolong the route and the time of the air passing through the water chamber. Therefore, the air can be saturated with moisture sufficiently, thereby increasing the air humidity.

Besides, when the air divided into two parts to each respectively enter the first tube and the second tube, an audio frequency caused by the air is changed due to a change of a cross sectional area. When the air from the second tube flows toward the air from the first tube, the two parts of the air hit with each other and then converge, thereby changing the audio frequency again and eliminating audio energies of the two parts of the air. The partition changes the audio frequency and eliminates audio energies of the air with different audio frequencies by the hit again and again in order to make multiple parts of the air in different portions of the water chamber have different audio frequencies, thereby reducing the resonance to eliminate noise.

In addition, because the first tube is shorter than the second tube, the first tube can relieve pressure to prevent the water in the water chamber from flowing back when the second tube is submerged by water.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a water chamber for a respiratory therapy apparatus in accordance with the present invention;

FIG. 2 is another perspective view of the water chamber for a respiratory therapy apparatus in FIG. 1;

FIG. 3 is an exploded perspective view of the water chamber for a respiratory therapy apparatus in FIG. 1;

FIG. 4 is a perspective view of a partition of the water chamber for a respiratory therapy apparatus in FIG. 1;

FIG. 5 is another perspective view of the partition of the water chamber for a respiratory therapy apparatus in FIG. 1;

FIG. 6 is an operational top view of the water chamber for a respiratory therapy apparatus in FIG. 1, showing a route of the air;

FIG. 7 is an operational front view of the water chamber for a respiratory therapy apparatus in FIG. 1, showing a route of the air; and

FIG. 8 is an operational bottom view of the partition of the water chamber for a respiratory therapy apparatus in FIG. 1, showing a route of the air.

### DETAILED DESCRIPTION OF THE PREFERRED EMBODIMENTS

With reference to FIGS. 1 to 3, a water chamber for a respiratory therapy apparatus with the present invention comprises a base casing 10, a cover casing 20, an inner space, and a partition 30.

The base casing 10 has a top opening. In a preferred embodiment, the base casing 10 is substantially semicircular in horizontal section. Alternatively, the base casing 10 may be in any other shape.

The cover casing 20 is mounted on the top opening of the base casing 10 and has a casing inlet 21 and a casing outlet 22. In a preferred embodiment, the cover casing 20 corresponds to the base casing 10 in horizontal section. The casing inlet 21 is formed through a sidewall of the cover casing 20. The casing outlet 22 is formed through a top of the cover casing 20. The casing inlet 21 and the casing outlet 22 are respectively adjacent to opposite sides of the cover casing 20. The cover casing 20 has a casing panel 23 formed on an inside of the top of the cover casing 20 as shown in FIGS. 6 and 7. Alternatively, the casing inlet 21 and the casing outlet 22 may be formed through other portions of the cover casing 20. For example, the casing inlet 21 may be formed through the top of the cover casing 20, and the casing outlet 22 may be formed through the side wall of the cover casing 20.

The inner space is formed between the base casing 10 and the cover casing 20.

With reference to FIGS. 3 to 7, the partition 30 is mounted between the base casing 10 and the cover casing 20, and divides the inner space between the base casing 10 and the cover casing 20 into an upper space and a lower space. The casing panel 23 of the cover casing 20 abuts the partition 30 and divides the upper space into an inlet space and an outlet space. The casing inlet 21 of the cover casing 20 communicates with the inlet space. The casing outlet 22 of the cover casing 20 communicates with the outlet space.

The partition 30 has a panel body 31, a first tube 32, a second tube 33 and a panel outlet 36. In a preferred embodiment, the partition 30 further has a dividing panel 34, a separating panel 35, a block panel 37, multiple supporters 38 and multiple outlet channels 39.

The panel body 31 has an inlet end and an outlet end. The inlet end corresponds to the casing inlet 21 of the cover casing 20. The outlet end corresponds to the casing outlet 22 of the cover casing 20.

The first tube 32 and the second tube 33 are formed on a bottom surface of the panel body 31. The first tube 32 has a top opening and a bottom opening to communicate with the inlet space and the lower space. The second tube 33 has a top opening and a bottom opening to communicate with the inlet space and the lower space. The first tube 32 is shorter than the second tube 33. The first tube 32 is nearer to the inlet end than the second tube 33. The second tube 33 is nearer to the outlet end than the first tube 32. In a preferred embodiment, the first tube 32 and the second tube 33 are both substantially rectangular, are arranged side by side, and are parallel to each other.

The dividing panel 34 is formed on a top surface of the panel body 31 and is in the inlet space. The dividing panel 34 extends from edges of the top openings of the first tube 32 and the second tube 33, and extends to the inlet end of the panel body 31 and the casing inlet 21 of the cover casing 20. In a preferred embodiment, the dividing panel 34 is curved and substantially corresponds to the semicircular base casing 10 in radian.

With reference to FIGS. 5, 7 and 8, the separating panel 35 is formed on the bottom surface of the panel body 31 and is in the lower space. The separating panel 35 extends from walls of the first tube 32 and the second tube 33, and extends to the inlet end of the panel body 31. In a preferred embodiment, the separating panel 35 is curved, and a radian of the separating panel 35 is equal to a radian of the dividing panel 34.

With reference to FIGS. 3 to 7, the panel outlet 36 is formed through the panel body 31 to communicate with the outlet space and the lower space. The panel outlet 36 is adjacent to the outlet end of the panel body 31 and is disposed below the casing outlet 22 of the cover casing 20.

The block panel 37 is mounted above the panel outlet 36.

The supporters 38 connect between an edge of the panel outlet 36 and the block panel 37.

Each outlet channel 39 is formed between two adjacent supporters 38, the edge of the panel outlet 36, and the block panel 37. In a preferred embodiment, the block panel 37 is inclined relative to the panel body 31, and distances between each two adjacent supporters 38 are different. Thus, cross sectional areas of the outlet channels 39 are different.

To assemble the water chamber as described, the casing inlet 21 of the cover casing 20 connects to an air output device. The casing outlet 22 of the cover casing 20 connects to an end of a connecting tube. The other end of the connecting tube connects to a mask.

With reference to FIGS. 3 to 6, when the water chamber is in use, air from the air output device enters the inlet space from the casing inlet 21 of the cover casing 20. Then, the air hits an end of the dividing panel 34 of the partition 30 and is divided into two parts. One part of the air flows along an outer sidewall of the dividing panel 34, and then soon flows to the top openings of the first tube 32 and the second tube 33. The other part of the air flows along an inner sidewall of the dividing panel 34, flows to another side of the dividing panel 34, hits the casing panel 23 and turns, and then flows to the openings of the second tube 33 and the first tube 32. The two parts of the air hit each other and converge at the top opening of the first tube 32 and the second tube 33, and then flow downward to the first tube 32 and the second tube 33. During the above process, because the two parts of the air pass through routes with different lengths, the two parts of the air have different speeds and therefore have different audio frequencies as well. The two parts of the air with different audio frequencies hitting each other can generate air with a new audio frequency and mutually eliminate audio energies of the two parts of the air, thereby reducing the resonance caused by the air to eliminate noise. Besides, a total cross sectional area of the top openings of the first tube 32 and the second tube 33 is different from cross sectional areas of the routes of the two parts of the air, such that speed and audio frequency of the convergent air can be further changed.

With reference to FIGS. 3, 5, 7 and 8, the convergent air enters the first tube 32 and the second tube 33, and is divided into two parts again. Each part of the air enters the lower space via the first tube 32 and the second tube 33. Because the second tube 33 is longer than the first tube 32, the part of the air from the second tube 33 passes through a relative longer route, which makes the speed of the part of the air relatively slower. According to Bernoulli's Principle, air with slower speed has higher pressure. Thus, the part of the air from the second tube 33 with relative higher pressure flows toward the part of the air from the first tube 32 with relative lower pressure. Therefore, the two parts of the air converge again, and the flowing direction is changed toward the inlet end. Afterward, the convergent air flows along an inside surface of the base casing 10 and the separating panel 35 to flow around by 180 degrees and then flows upward to the panel outlet 36.

With reference to FIGS. 3, 5, and 7, as soon as the air enters the outlet space from the panel outlet 36, the air hits the block panel 37 and is divided into multiple parts. The multiple parts of the air are turned transversely and respectively to flow to the outlet channels. Then, the multiple parts of the air turn upward and converge Finally, the air flows to the casing outlet 22, the connecting tube, and the mask in sequence. During the above process, the air directly hits the block panel 37, and is forced to change the direction almost vertically. Thus, the audio frequency caused by the air is changed again. On the other hand, the cross sectional areas of the outlet channels 39 are different from each other such that the multiple parts of the air passing through the outlet channels 39 have different speeds. Therefore, when the multiple parts of the air with different speeds converge, the convergent air has a new audio frequency.

The partition as described divides the air into multiple parts to change the audio frequency and the speed of the air, and then makes the multiple parts of the air hit each other and converge to form an air with a new audio frequency and to eliminate the audio energies of the air with different audio frequencies. As the above flowing movement is repeated again and again, the partition makes the air in different portions of the water chamber have different audio frequencies, thereby reducing the resonance to eliminate noise.

With reference to FIGS. 3, 6 to 8, with the dividing panel 34, the casing panel 23, the first tube 32, the second tube 33 longer than the first tube 32, the separating panel 35 and the block panel 37, the flowing route of air are made winding and indirect such that the air has to take longer time to pass through the water chamber. Therefore, the air can be saturated with moisture sufficiently, thereby increasing the air humidity.

With reference to FIGS. 3, 5 and 7, in addition, if breathing of the user generates pressure and makes the water in the lower space flow backward to the inlet space from the second tube 33, the water spilling out of the second tube 33 may flow downward to the lower space from the first tube 32, thereby preventing the air output device from being damaged by the water.

With reference to FIGS. 4, 5, and 7, the inclination of the block panel 37 further facilitates forming the outlet channels 39 with different cross sectional areas to generate air with different speeds and audio frequencies. In addition, if water flows into the outlet space from the casing outlet 22 accidentally, the inclined block panel 37 disposed below the casing outlet 22 can prevent the water from staying on the block panel 37.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A partition of a water chamber for a respiratory therapy apparatus comprising:
a panel body having
an inlet end; and
an outlet end;
a first tube formed on a bottom surface of the panel body, and having
a top opening; and
a bottom opening;
a second tube formed on the bottom surface of the panel body, and having
a top opening; and
a bottom opening; and
a panel outlet formed through the panel body and being adjacent to the outlet end;
wherein the first tube is shorter than the second tube; the first tube is nearer to the inlet end than the second tube; and the second tube is nearer to the outlet end than the first tube.

2. The partition as claimed in claim 1, wherein the first tube and the second tube are arranged side by side and are parallel to each other.

3. The partition as claimed in claim 1 further comprising a dividing panel formed on a top surface of the panel body, extending from edges of the top openings of the first tube and the second tube, and extending to the inlet end.

4. The partition as claimed in claim 2 further comprising a dividing panel formed on a top surface of the panel body, extending from edges of the top openings of the first tube and the second tube, and extending to the inlet end.

5. The partition as claimed in claim 3, wherein the dividing panel is curved.

6. The partition as claimed in claim 4, wherein the dividing panel is curved.

7. The partition as claimed in claim 1 further comprising a separating panel formed on the bottom surface of the panel body, extending from walls of the first tube and the second tube, and extending to the inlet end.

8. The partition as claimed in claim 6 further comprising a separating panel formed on the bottom surface of the panel body, extending from walls of the first tube and the second tube, and extending to the inlet end.

9. The partition as claimed in claim 7, wherein the separating panel is curved.

10. The partition as claimed in claim 8, wherein the separating panel is curved, and a radian of the separating panel is equal to a radian of the dividing panel.

11. The partition as claimed in claim 1 further comprising:
a block panel mounted above the panel outlet;
multiple supporters connected between an edge of the panel outlet and the block panel; and
multiple outlet channels, each outlet channel formed between two adjacent supporters, the edge of the panel outlet, and the block panel.

12. The partition as claimed in claim 10 further comprising:
a block panel mounted above the panel outlet;
multiple supporters connected between an edge of the panel outlet and the block panel; and
multiple outlet channels, each outlet channel formed between two adjacent supporters, the edge of the panel outlet, and the block panel.

13. The partition as claimed in claim 11, wherein the block panel is inclined relative to the panel body.

14. The partition as claimed in claim 12, wherein the block panel is inclined relative to the panel body.

15. A water chamber with a partition as claimed in claim 1 for a respiratory therapy apparatus, the water chamber comprising:
a base casing having a top opening;
a cover casing mounted on the top opening of the base casing and having
a casing inlet;
a casing outlet; and
a casing panel;
an inner space formed between the base casing and the cover casing; and
the partition mounted between the base casing and the cover casing, and dividing the inner space into an upper space and a lower space;
wherein the casing panel of the cover casing divides the upper space into an inlet space and an outlet space; the casing inlet of the cover casing communicates with the inlet space and corresponds to the inlet end of the panel body; the casing outlet of the cover casing communicates with the outlet space and corresponds to the outlet end of the panel body; the first tube and the second tube communicate with the inlet space and the lower space; the panel outlet communicates with the outlet space and the lower space and corresponds to the casing outlet.
